(19) United States
(12) Patent Application Publication
(10) Pub. No.: US 2024/0269317 A1
(43) Pub. Date: Aug. 15, 2024

(54) TARGETED ENCAPSULATION STRATEGIES FOR TREATMENT MODALITIES

(71) Applicant: NUTARIA LTD., Beer Sheba (IL)

(72) Inventor: Omer PORAT, Beer Sheva (IL)

(21) Appl. No.: 18/601,302

(22) Filed: Mar. 11, 2024

Related U.S. Application Data
(63) Continuation of application No. PCT/IL2022/051014, filed on Sep. 22, 2022.
(60) Provisional application No. 63/248,448, filed on Sep. 25, 2021.

(51) Int. Cl. A61K 47/69; A61K 47/54; A61K 47/55

(52) U.S. Cl. CPC A61K 47/6911; A61K 47/544; A61K 47/55

(57) ABSTRACT

Compositions useful for delivery of active pharmaceutical ingredients (APIs) and active nutraceutical ingredients (ANIs) to a targeted tissue or a targeted cell of a mammalian body, comprising a complex of at least one nanoparticle; at least one API; the API is contained by the nanoparticle; at least one nutraceutical ligand, the nutraceutical ligand is configured to specifically bind to the targeted tissue or a targeted cell; wherein the API or the ANI is activated at the targeted tissue or at a targeted cell by the mediation of the nutraceutical ligand.

### CROSS REFERENCE TO RELATED APPLICATIONS

This application is a Bypass continuation of PCT Patent Application No. PCT/IL2022/051014 having International filing date of Sep. 22, 2022, which claims the benefit of priority of U.S. Provisional Patent Application No. 63/248,448, filed Sep. 25, 2021, the contents of which are all incorporated herein by reference in their entirety

### FIELD OF THE INVENTION

This invention is in the field of targeted and enriched nutraceutical-based treatments/medications.

### BACKGROUND OF THE INVENTION

There are a large number of chronic or long term health conditions that are challenging to treat. Diseases such as neuro-degenerative disorders, cancer, diabetes, IBD have shown to possess an elusive pharmaco-therapeutic target and are highly treatment-resistant.

Most pharmacological treatments/intervention/therapies have been shown to cause a wide variety of adverse and side effects. Many pharmacological treatments consist of chemicals that show extensive side effects, often related to interactions between the chemicals and other pharmacological targets. These other pharmacological targets can be expressed in other tissues or organs. For example, the endocannabinoid system is expressed in the central nervous system and digestive tract (CB1) in addition to the peripheral nervous system, immune system and digestive tract (CB2), thus the biological effect of various cannabinoids depends on the affinity to the different cannabinoid receptors. Additional mechanisms leading to side effects are variations in pharmacokinetics or drug-drug interactions. In the case of Parkinson's disease, for example, some of the side effects are related to the interaction of L-dopa (the therapeutic chemical) with receptors in the peripheral nervous system whereas, in the central nervous system (CNS), L-dopa is selectively metabolized to dopamine.

One solution for improving the efficacy of the therapeutics and limiting side effect is to selectively target the pharmacological composition to the tissue or organ affected by the disorder. Selective delivering of the therapeutic agent to a target tissue or organ can enable the use of a lower dosage, further lowering the chance of adverse effects.

There is an unmet need for a targeted therapeutic for the treatment of chronic and long term medical conditions.

### SUMMARY

The present invention discloses a composition for tissue-specific and cell-specific delivery of active ingredients which may be active pharmaceutical ingredients (API) or active neutraceutical ingredients (ANI). The specific delivery is obtained by means of a specific nutraceutical ligand, thus enabling the delivery of medicaments and food supplements to specific sites, therefore providing a long-needed aid to subjects suffering from various ailments.

It is hence one object of the invention to disclose a composition for delivering an active pharmaceutical ingredient (API) to a targeted tissue or a targeted cell of a mammalian body, comprising a complex comprising of: at least one nanoparticle; at least one API; the API is contained by the nanoparticle; at least one nutraceutical ligand, the nutraceutical ligand is configured to specifically bind to the targeted tissue or the targeted cell; wherein the API is activated at the targeted tissue or at the targeted cell by the mediation of the nutraceutical ligand.

It is another object of the invention to disclose the composition as defined above, wherein the composition further comprises a linker that binds the at least one nutraceutical ligand to the at least one nanoparticle.

It is another object of the invention to disclose the composition as defined in any of the above, wherein the nutraceutical is a targeting nutraceutical.

It is another object of the invention to disclose the composition as defined in any of the above, wherein the nanoparticle is selected from the group consisting of a liposome, a micelle, a solid-lipid nanoparticle, a cyclodextrin nanoparticle, a dendrimer, a polymeric nanoparticle, a micro/nano-emulsion, and any combinations thereof.

It is another object of the invention to disclose the composition as defined in any of the above, wherein the micelle is a mono-layer or a bi-layer construction.

It is another object of the invention to disclose the composition as defined in any of the above, wherein the diameter of the nanoparticle is in the range of 10-250 nm.

It is another object of the invention to disclose the composition as defined in any of the above, wherein the API is bound to the nanoparticle via a chemical or a physical bond.

It is another object of the invention to disclose the composition as defined in any of the above, wherein the API is contained within the mono-layer or bi-layer, contained between layers of the bi-layer, or bound to an external layer of the mono-layer or bi-layer.

It is another object of the invention to disclose the composition as defined in any of the above, wherein the chemical or physical bond is selected from the group consisting of a covalent bond, a hydrogen bond, a wan der Waals bond, a hydrophobic bond, an electrostatic bond, a London bond, a ionic bond, a salt bridge, adsorbtion, and any combination thereof.

It is another object of the invention to disclose the composition as defined in any of the above, wherein the composition is adapted to release the API at the targeted tissue.

It is another object of the invention to disclose the composition as defined in any of the above, wherein the API is selected from a group consisting of a nutraceutical, a small molecule, a food supplement, a protein, a hormone, a peptide, and a fatty acid.

It is another object of the invention to disclose the composition as defined in any of the above, wherein the nutraceutical is an active nutraceutical ingadient (ANI).

It is another object of the invention to disclose the composition as defined in any of the above, wherein the ANI is selected from a group consisting of a neuro-active nutraceutical, an onco-active nutraceutical, an endo-active nutraceutical, a gastro-active nutraceutical, and any combination thereof.

It is another object of the invention to disclose the composition as defined in any of the above, wherein the ANI is curcumin.

It is another object of the invention to disclose the composition as defined in any of the above, wherein the API is generally recognized as safe (GRAS).

It is another object of the invention to disclose the composition as defined in any of the above, wherein the nutraceutical ligand is selected from a group consisting of a neuro-targeting compound, a tumor-targeting compound, a gastric targeting compound, an endo (pancreatic)—targeting compound, a cardiovascular targeting compound, a kidney targeting compound, an inflammation targeting compound, and any combination thereof.

It is another object of the invention to disclose the composition as defined in any of the above, wherein the neuro-targeting compound is selected from a group consisting of caffeine, theobromine (xantheose), paraxanthine, theophylline, 1,3,7-trimethyluric acid, guarana (Paullinia cupana, HBK), phenethylamine (PEA, chocolate), tryptamine (Acacia), and any combinations thereof.

It is another object of the invention to disclose the composition as defined in any of the above, wherein the neuro-targeting compound is selected from a group consisting of baicalein, wogonin, oroxylin A, and any combination thereof.

It is another object of the invention to disclose the composition as defined in any of the above, wherein the nutraceutical ligand is generally recognized as safe (GRAS).

It is another object of the invention to disclose the composition as defined in any of the above, wherein the composition is configured to achieve at least one of the following: a higher concentration of said API at the target tissue; a lower concentration of said API in non-targeted tissue; a higher activity of said API at the target tissue; an extended activity of said API at said target tissue; an increased delivery of the API to a target cell.

It is another object of the invention to disclose the composition as defined in any of the above, wherein the delivery mechanism of the API is characterized as being an active or a passive uptake mechanism.

It is another object of the invention to disclose the composition as defined in any of the above, wherein the uptake mechanism of the API is defined as the uptake of the at least one nanoparticle containing the at least one API, or of the at least one API released from the at least one nanoparticle.

It is another object of the invention to disclose the composition as defined in any of the above, wherein the uptake mechanism of the at least one nanoparticle containing the at least one API is endocytosis.

It is another object of the invention to disclose the composition as defined in any of the above, wherein the mode of the endocytosis is selected from the group consisting of receptor-mediated endocytosis, clathrin-mediated endocytosis, non-mediated endocytosis, caveolae, pinocytosis and phagocytosis.

It is another object of the invention to disclose the composition as defined in any of the above, wherein the active uptake mechanism is characterized as being facilitated by one of the group consisting of transporters, pumps and channels.

It is another object of the invention to disclose the composition as defined in any of the above, wherein the passive uptake mechanism is selected from the group consisting of diffusion, facilitated diffusion and filtration.

It is another object of the invention to disclose the composition as defined in any of the above, wherein the uptake is selected from the group consisting of secondary active transport, coupled transport or co-transport.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide a further understanding of the invention and are incorporated in and constitute a part of this specification, illustrate embodiments of the invention and together with the description serve to explain the principles of the invention wherein:

FIG. 1—a schematic representation of an embodiment of the composition.

FIG. 2—a schematic representation of the binding activity of an embodiment of the composition.

FIG. 3—results of a toxicity experiment of an embodiment of the composition.

FIGS. 4a-e—results of API delivery experiment of an embodiment of the composition.

FIGS. 5a-b—results of API delivery experiment of another embodiment of the composition.

FIGS. 6a-c—imaging of API delivered by an embodiment of the composition in target cells.

### DETAILED DESCRIPTION OF THE PREFERRED EMBODIMENT

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide compositions and methods.

It is core objective the present invention to disclose and provide a platform for the targeted delivery of an active pharmaceutical ingredient (API) guided to the targeted mammalian cell, tissue or organ by a nutraceutical ligand. The platform would provide a nanoparticle having a shell decorated with at least one nutraceutical targeting compound or nutraceutical ligand and a core comprising at least one active pharmaceutical ingredient or nutraceutical compound. The nutraceutical ligand binds to specific receptors on the mammalian cell, tissue or organ.

In this application, the term “nanoparticle” (NP) relates to a particle between 1 and 250 nanometers (nm) in diameter, having a core surrounded by an interfacial layer (the shell). In some embodiments, the nanoparticle is selected from a liposome, a micelle, a solid-lipid nanoparticle, a cyclodextrin nanoparticle, a dendrimer, a polymeric nanoparticle, a micro/nano-emulsion and any combinations thereof.

In some embodiments, the NP is a vessel or a liposome, comprising a lipid bilayer or a single layer (a multilamellar liposome or a unilamellar liposome).

In one embodiment of the present application, the outer surface of the nanoparticle (the shell) is decorated, so as to include at least one targeting compound (a nutraceutical ligand), oriented so as to be available for binding to the target. The target could be a tissue, organ or specific cell.

The term ‘Nutraceutical’ was first devised in 1989 by Stephen Defelice, and it is defined as “any substance that is a food or a part of the food and provides medical or health benefits, including the prevention and treatment of disease”. These nutraceuticals including the isolated nutrients, dietary supplements (minerals, vitamins, amino acids, fatty acids, saccharides, probiotics, prebiotics, and herbs) and phytochemicals promised to have beneficial health effects besides the traditional nutrition values found in food. Nutraceuticals are commonly derived from food sources, plant matter, fungus, bacteria or microbes derived from a natural source, such as a plant or bacteria. The nutraceutical could be defined as an alkaloid, a phenol (or a polyphenol), a peptide, a saccharide, a terpene, phenolic, glycoside a fatty acid, a flavonoid, a phytosterol, and as tannins. The nutraceutical can serve as a primary or a secondary metabolite.

One of the major benefits of nutraceuticals over pharmaceuticals include a much faster “to market” period, lesser or no adverse effects, economically affordable, easily available, multiple therapeutic effects, and enables long term consumption without toxicity or titration concerns.

The decoration is achieved by any type of modification of the shell (chemically bonding to the shell, chemically coordinating with the shell, forming a part of the shell, polymerized into the shell, and so forth) or by modifying a nutraceutical ligand so as to bind to the shell. In some embodiments, the complex comprises a linker that binds to the nutraceutical ligand and to the NP. In a prominent non limiting example of the present invention, the nutracutical ligand is caffeine, and the API is an ANI, namely curcumin.

The invention further provides a nanoparticle-based composition comprising a nanoparticle-shell decorated with at least one neuro-targeting nutraceutical and a core comprising at least one neuro-active nutraceutical, for use in the treatment of at least one neurodegenerative disease, neoplastic disease, digestive disease, endocrine disorders, kidney disorders, cardiovascular diseases and any symptom thereof.

An active pharmaceutical ingredient (API) is defined as “Any substance or combination of substances used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings” (WHO, 2011). The API can be classified according to its structure (such as antibodies), its therapeutic target (such as analgesics) or its biological effect (such as Non-steroidal anti-inflammatory drug—NSAID). The API can be a small molecule (such as NSAID) or a macromolecule (such as antibodies). The API can be from a natural source (such as a natural product) or produced in a semi-synthetic or synthetic manor.

In some embodiments, the API is delivered to the target tissue or cell through an active or a passive uptake mechanism. In some embodiments, the uptake mechanism is of the whole API-contained NP, such as endocytosis. The endocytosis can be mediated, such as receptor-mediated endocytosis (also known as clathrin-mediated endocytosis), or non-mediated (such as caveolae, pinocytosis, and phagocytosis). In some embodiments, the API is released by the NP into the extracellular matrix and the API uptake is of a released (un-bound) API. The uptake can be an active mechanism (such as transporters, pumps, and channels) or passive (such as diffusion, facilitated diffusion and filtration). In some embodiments, the transport is secondary active transport (known as coupled transport or cotransport).

In some embodiments, the API is a nutraceutical. This API is referred to as active nutraceutical ingradient (ANI).

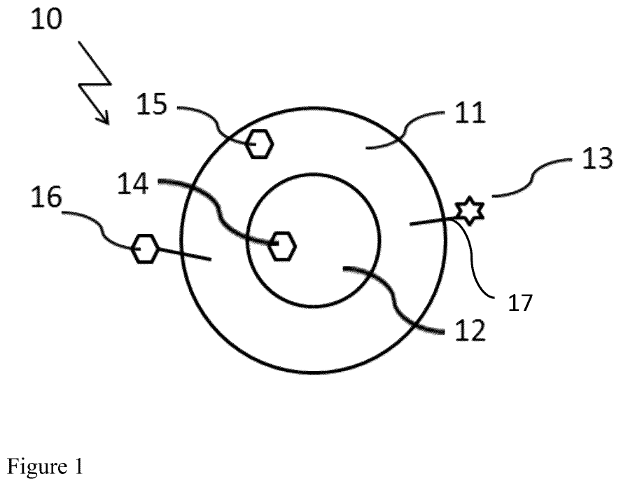

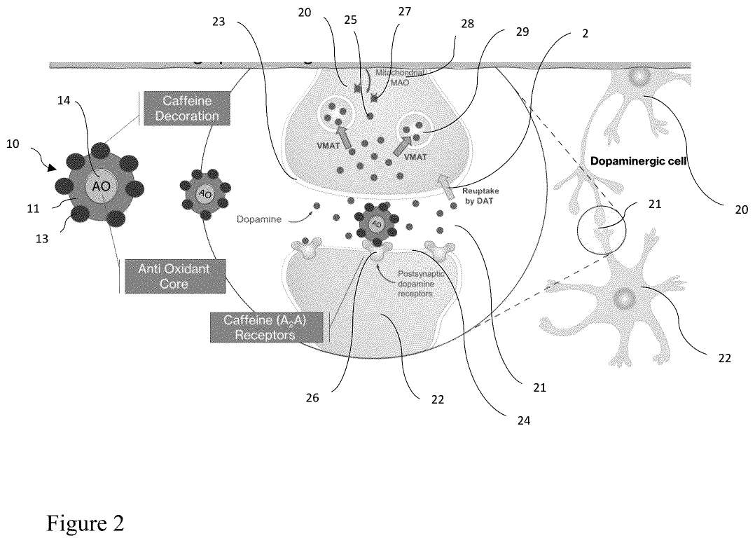

Reference is made to FIG. 1, showing an embodiment of the composition, comprising a nanoparticle 10, the nanoparticle 10 comprising a bilayer liposome 11 with an internal volume 12. The targeting compound 13 is bound to the outer shell via a linker 17, while an API is either contained within the internal volume 14, within the bilayer 15 or bound to the external shell 16. Reference is made to FIG. 2, showing schematic representation of the binding activity of an embodiment of the composition. The composition comprises a nanoparticle 10 decorated with caffein ligands 13 on the outer surface of the bi-layer 11 of the nanoparticle 10. The nanoparticle 10 contains an anti-oxidative neuro-active API 14. The nanoparticle 10 binds the target cell 22 thereof, via the caffein ligand 13. This target cell 22 is a postsynaptic cell comprising postsynaptic dopamine receptors 26 on the postsynaptic membrane 24 thereof. Dopamine molecules 25 are released by a presynaptic cell 20 via vesicular monoamine transporters (VMAT) 29, into a synapse 21. The excess molecules 25 are later reuptaken from the synapse 21 by a dopamine transporter (DAT) 2, back into the presynaptic cell 20, where they are catabolized 27 by mitochondrial monoamine oxidase (MAO) 28. In other embodiments, nanoparticles can be decorated by DAT-binding ligands.

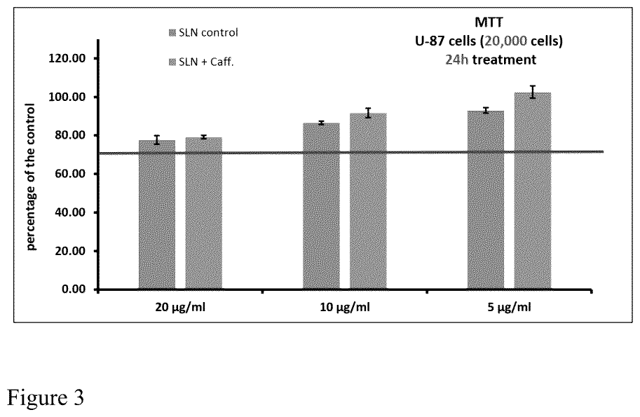

Reference is made to FIG. 3, showing results of an in-vitro toxicity experiment of an embodiment of the composition. Human Astrocytes (U-87 MG cell line) were exposed to solid lipid nanoparticles (SLNs) 10 decorated by caffein ligands 13, and containing the ANI curcumin 14. Cell viability was determined using 3-(4,5-dimethylthiazol-2-yl)-2,5-diphenyltetrazolium bromide (MTT) assays. After overnight stabilization, cells were exposed to varying concentrations of the SLNs 10. After 24 hours, the madia was removed, and all cells were incubated with cerum-free media containing MTT for 3-4 hours in the incubator. The MTT purple crystals formed by the viable cells were diluted using isopropanol containing 0.04 mol/L HCl. Optical density was measured at 570 nm. Data is shown as percentage of untreated cells (control). Data are mean ±SE (n=4). No toxic effect was observed following the SLN 10 treatments with the above mentioned concentrations. The cell viability of the treated cells was even higher than of the control cells.

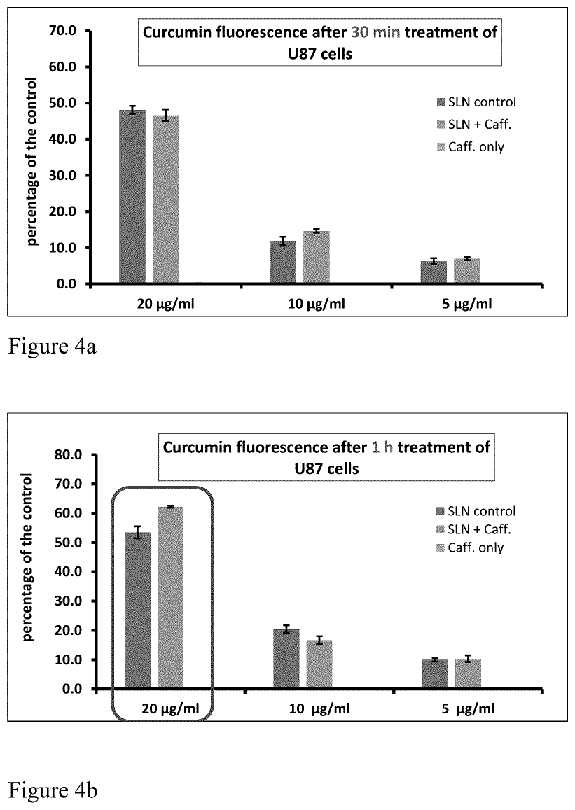

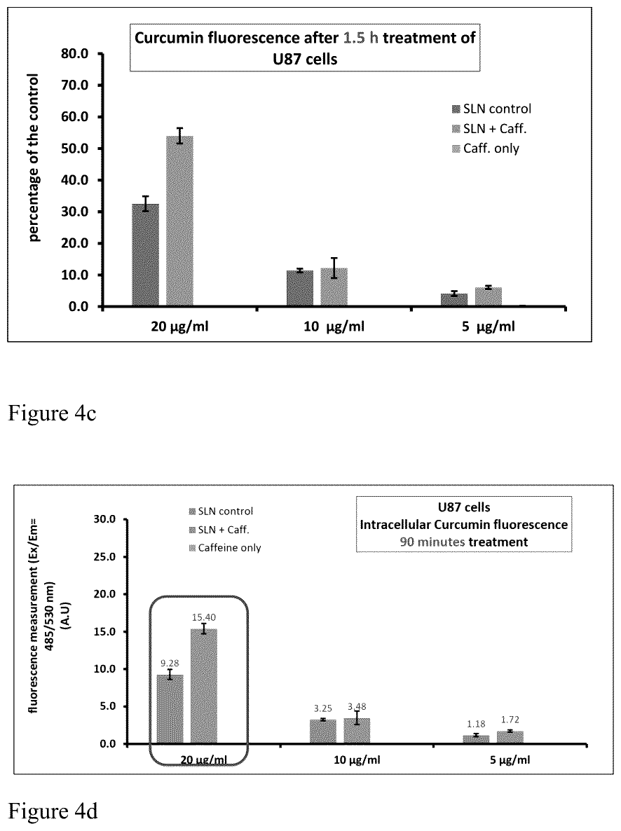

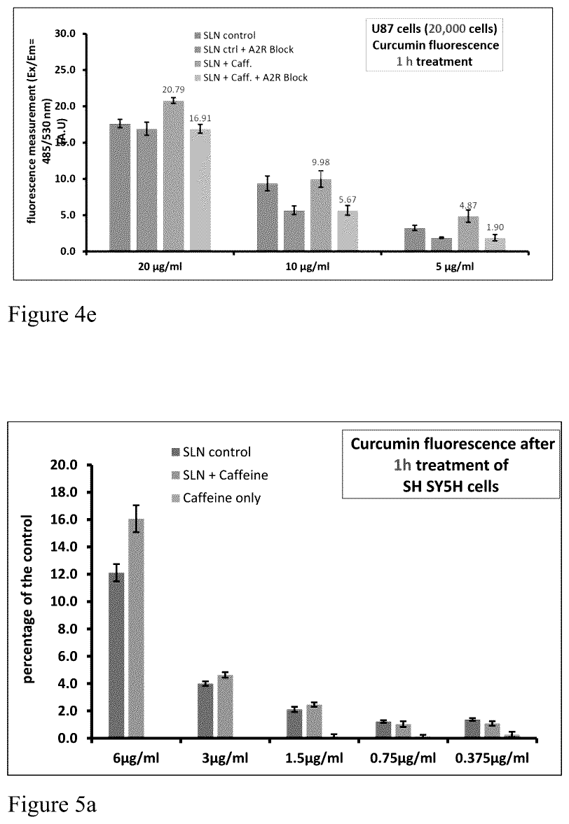

Reference is made to FIGS. 4a-e, showing results of an in-vitro curcumin delivery experiment of an embodiment of the composition. Human Astrocytes (U-87 MG cell line) were exposed to solid lipid nanoparticles (SLNs) 10 decorated by caffein ligands 13, and containing the ANI curcumin 14. FIGS. 4a-c depict different time points of the same experiment. Cells were seeded and grown for 24 h before treatment. Each time point was measured in a different plate. Media was replaced with fresh media containing the different treatments. After the treatment duration, cells were washed using PBS X1 and lysed using RIPA buffer. After 5 min of agitation, fluorescence was measured using curcumin specific wavelength of curcumin 14 (Ex/Em=485/530 nm). The results are shown as percentage of a curcumin sample (served as control). Data are mean ±SE (n=4). As shown in FIG. 4a, no significant difference between SLNs 10 decorated with caffein 13 and ligand-less SLNs 10 were spotted after 0.5 hour. However, after 1 h (FIGS. 4b) and 1.5 h (FIG. 4c) of treatment, a significant increase in the curcumin fluorescence was observed following SLN+Caffeine sample, as compared to the ligand-less SLN control sample and to SLN-less caffeine sample. This indicates that the coating of the sample with caffeine 13 augments the intracellular content of curcumin 14 at 1.5 hours. An experiment similar to FIG. 4c is depicted on FIG. 4d. In both experiments, measurement were taken after 1.5 hours of treatment. The only difference is the absolute amount of curcumin 14 in the cells. However, the relations between the results of the different treatments are similar in FIG. 4c and FIG. 4d. FIG. 4e depicts results of an in-vitro curcumin delivery experiment of an embodiment of the composition, which is similar to the experiment depicted in FIG. 4d, with an addition of another control variable: A2R receptor blocker, which was used since caffeine 13 is an antagonist to A2R receptors. The results indicates that indeed the blocking of A2R receptors with an antagonist, reduces the curcumin content to the same levels as the SLN control (in the concentration of 20 mg/ml).

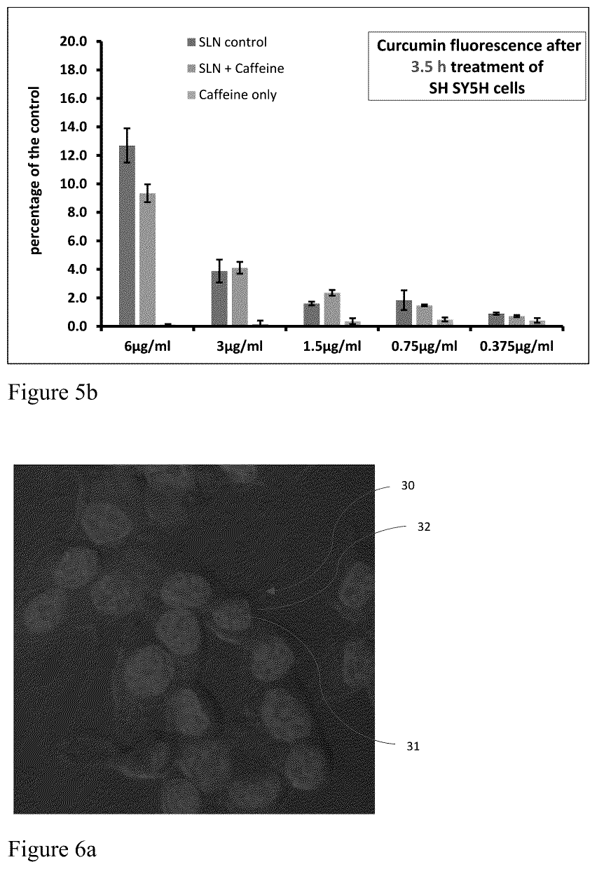

Reference is made to FIGS. 5a-b, showing results of an in-vitro curcumin delivery experiment of another embodiment of the composition. Human Neuroblastoma cells (SH Sy5Y cells cell line) were exposed to solid lipid nanoparticles (SLNs) 10 decorated by caffein ligands 13, and containing the ANI curcumin 14. FIGS. 5a-b depict different time points of the same experiment. As depicted in FIG. 5a, after 1 hour, a significant increase in the curcumin 14 fluorescence was observed following SLN 10+Caffeine 13 sample, as compared to the SLN 10 control sample and to curcumin 14 sample. However, as depicted in FIG. 5b, after 3.5 hours of treatment, the fluorescence of the SLN 10 control sample and the curcumin 14 sample remained at the same levels, as opposed to the SLN 10+Caffeine 13 sample, which diminished considerably. This effect might be due to intra-cellular metabolism of the curcumin present in the SLN 10+Caffeine 13 sample.

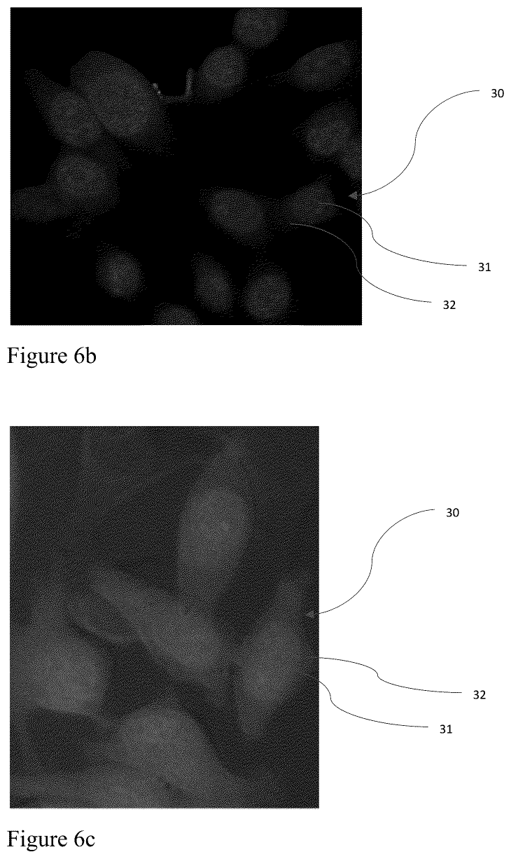

Reference is made to FIGS. 6a-c, showing fluorescence imaging of Human Astrocytes (U-87 MG cell line) exposed to solid lipid nanoparticles (SLNs) 10, the SLNs 10 being decorated by caffein ligands 13 and containing the ANI curcumin 14. The cells 30 were seeded in a 24-well plate at a density of 8×10\hat{ }4 cells/well and were incubated for overnight stabilization. After the treatment, the cells 30 were washed with PBS X1 and fixate using 3% paraformaldehyde for twenty minutes. After second washing with PBS, the cellular nucleus 31 was stained using Hoechst 33342. The analysis was performed using Nikon's AIR+confocal microscope in The Hebrew University of Jerusalem Interdepartmental Equipment Facility. In this imaging assay, the green fluorescence in the pictures indicates the presence of curcumin 32 in the cytoplasm, while the blue color indicates the nuclei 31 of the cells. FIG. 6a depicts untreated control cells, FIG. 6b depicts cells exposed linandless control SLNs 10 for 1.5 hours, and FIG. 6c depicts cells exposed to SLNs 10 decorated by caffeine ligands 13 for 1.5 hours. A significantly higher curcumin 32 staining can be observed in the SLN 10 +Caffeine 13 treatment at 1.5 hours, compared to SLN 10 control and untreated cells 30. In untreated cells 30, green color is not detectable. In SLN 10 control cells 30, certain amount of green color is detectable around the nuclei 31 of the cells 30. In caffeine-decorated cells 30, the green color is so strong it even interferes with the blue color of the nucleus 31. Numerical analysis of the fluorescence images will be provided later.

Targeting activity: In this application, the term “targeting nutraceutical” refers to a compound that is derived from a natural source and has been clinically substantiated to contribute in delivering a nanoparticle of the invention in a manner that increases the local concentration of the nanoparticle in specific organ or tissue, such as in the central nervous system (CNS) or parts of it, relative to other parts of the subject body. The targeting nutraceutical can also be referred to as a bio-carrier, as its ability to ‘carry’ the nanoparticle conjugate to the target organ, tissue or cell. The goal of a targeted delivery system of the invention is to provide a targeted, localized, extended/long-effect interaction with the target. The targeting nutraceutical can be targeted to various systems and disorders, such as the central or peripheral nervous system (such as Parkinson's disorder, Alzheimer's disease or multiple sclerosis), neoplastic growths (tumor), endocrine system (such as diabetics) or the gastrointestinal system (such as gastric ulcers or IDB).

The advantages to the targeted delivery relate to the localized concentration of the medication in the tissues of interest while the reduction in the relative concentration of the medication in the remaining tissues. This strategy can lead to various beneficial effects:
a. Lower doses of active compounds;
b. A reduction in the frequency of the dosages taken by the subject;
c. A more consistent pharmacological effect;
d. A reduction of harmful side-effects by reducing and controlling changes in circulating levels of the nanoparticle of the invention.

In some embodiments, the targeting compound is a neuro-targeting nutraceutical:

In some embodiments, the neuro-targeting nutraceutical is selected from caffeine and its four dimethylxanthines-metabolites, including, Theobromine (xantheose), Paraxanthine, Theophylline, and 1,3,7-Trimethyluric acid, guarana (Paullinia cupana, HBK), Phenethylamine (PEA, chocolate), Tryptamine (Acacia) and any combinations thereof. Caffeine is an adenosine receptor antagonist (A2AR), which is expressed in PD target sites (Svenningsson et al. 1999, Madreira et al., 2017; Rivera-Oliver et al., 2014). In some embodiments, the targeting nutraceutical is selected from theobromine (xantheose), a bitter alkaloid of the cacao plant, and its alkaloid analogs.

In some embodiments, the targeting compound is another group of neuro-targeting nutraceuticals, specifically targeting cell bodies of dopamine neurons: baicalein, wogonin, and oroxylin A. These neuro-targeting nutraceutical bind the same receptors as cocaine, and are therefore likely to be usefull in treatment of substance addictions.

Since dopamine receptors are mostly post-synaptic, DATs (dopamine transporters), are selected for targeting. DATs are membrane-spanning transporters that pump dopamine out of the synaptic cleft back into the cytosol. Each DAT is composed of twelve transmembrane domains (TMD) with a large extracellular loop between the third and fourth TMDs [Ben-Jonathan, Nira. Dopamine. CRC Press. Kindle Edition].

Unlike amino acid transporters, which are found on both neurons and glia, the expression of DAT is restricted to dopaminergic neurons, making DAT a well-accepted experimental and pathological marker for these neurons. At the cellular level, DAT is found in both dendrites and cell bodies of neurons in the substantia nigra and ventral tegmental areas.

In view of the above, the neuro-targeting nutraceutical, natural ligands for DAT are proposed as baicalein, wogonin, and oroxylin A, which are main active flavones in the medicinal herbs Scutellaria baicalensis and Scutellaria lateriflora. [Ji, Yeongscon, et al. “Neuroprotective effects of baicalein, wogonin, and oroxylin A on amyloid beta-induced toxicity via NF-κB/MAPK pathway modulation.” Molecules 25.21 (2020): 5087)].

In some embodiments, the targeting nutraceutical is an onco-targeting nutraceutical:

In some embodiments, the onco-targeting nutraceutical is selected from saccharides and sugar substitutes. In some embodiments, the targeting nutraceutical is a modified sugar, such as sucralose (chlorinated sucrose). In some embodiments, targeting nutraceutical is sugar alcohol such as sorbitol, xylitol, erythritol, mannitol, maltitol, lactitol, erythritol. In some embodiments the sugar substitute is Saccharin. The modified sugar or sugar alcohol can demonstrate slower metabolism and higher accumulation in the extravascular extracellular space (EES) of tumors.

In some embodiments, the targeting nutraceutical is an endocrine-targeting nutraceutical:

In some embodiments, the endocrine-targeting nutraceutical is selected from a group of insulin analogs and insulin receptor agonists/antagonists. The Insulin receptor is a transmembrane tyrosine kinase receptor that binds to insulin and insulin growth factors (I and II). In some embodiments, the insulin analog is Momordica charantia insulin receptor-binding protein or a peptide segment of the protein. In some embodiments, the insulin receptor can be activated by Biotin, Magnesium, Chromium, Gymnema s., Camellia s. and Vaccinium m. [see Covolo et al. (2017) BMC Public Health, 13, 777].

In some embodiments, the targeting nutraceutical is a gastro-targeting nutraceutical:

In some embodiments, the NP comprises a nutraceutical-based coating that lowers the biodiversity of the NP and increases the delivery of the NP to the intestine. In some embodiments, the coating lowers the solubility of the NP. The coating can be lipid or saccharide based. In some embodiments, the coating increases the resistance of the NP to the conditions in the gastrointestinal tract, such as the stomach or and the intestine.

In some embodiments, the gastro-targeting nutraceutical is a long-chain fatty acid (LCFA), such as oleic acid, linoleic acid, and linolenic acid, that binds to the Intestinal-fatty acid-binding protein (I-FABP) in small intestinal epithelial cell lines.

In some embodiments, the NP comprises a kidney or heart targeting nutraceutical. In some embodiments, the kidney or cardiovascular targeting compound is a low-molecular-weight peptide.

In some embodiments, the peptide is a Natriuretic peptide (or analog), that binds to the atrial natriuretic peptide receptor in the kidney or cardiovascular system [see Massimo et al. 2014. European Heart Journal, 35, 419-425].

In some embodiments, the targeting nutraceutical is an inflammation -targeting nutraceutical.

In some embodiments, the inflammation -targeting nutraceutical is Isoflavone (Phytoestrogen), in soy foods.

In some embodiments, the ligand attached to the NP is genistein or daidzein, two isoflavones from soy which affect on rheumatoid arthritis (RA).

One of the possible mechanisms of genistein and daidzein action is their estrogen-like effects. Genistein is structurally related to estrogen and binds estrogen receptors. It has already been demonstrated that estrogen-like sexual hormones improve and prevent RA status (McMurray 2001). It is also possible that the isoflavones exert their effects through a direct effect on cartilage metabolism. Both estrogen a and B receptors can be found in the cartilage of human joints, so cartilage may also be a target for modulators of estrogen receptors. Intra-articular injection of estrogen leads to the destruction of matrix collagen by destroying lactate dehydrogenase enzyme in chondrocytes. Estrogen-like isoflavones that bind to estrogen receptors and interfere with local estrogen function can be effective in osteoarthritis. [See Mohammad-Shahi, Majid et al. “Comparison of the effects of genistein and daidzein with dexamethasone and soy protein on rheumatoid arthritis in rats.” BioImpacts : BI vol. 1,3 (2011): 161-70.]

In some embodiments, the inflammation-targeting nutraceutical is flavonoids and other phytochemicals. Flavonoids and other phytochemicals from oregano were characterized for their agonist and antagonist PPARγ activities.

The peroxisome proliferator activated receptors (PPARs) are a subset of the nuclear receptor superfamily. Unlike the classical hormone-activated receptors such as the estrogen receptor, which is located in the cytoplasm and translocates to the nucleus after binding of the activating ligand, the PPAR receptors reside in the nucleus bound to DNA response element

Through their binding to PPARs—or directly by their binding to NF-KB and AP-1—dietary molecules can inhibit the inflammatory process.

Dietary phytochemicals with quite different structures and properties from the unsaturated fatty acids discussed so far, have been characterized from oregano by activity-based fractionation. Compounds were first identified as ligands using a PPARγ fluorescence-polarization assay and subsequently classified as agonist or antagonist, based on their ability to inhibit rosiglitazone-induced cellular responses.

In some embodiments, the NP is the dietary flavonoids quercetin, luteolin, rosmarinic acid and diosmetin . The dietary flavonoids were found to be PPARγ antagonists, naringenin and apigenin were modulators and biochanin A was found to be a PPARγ agonist [see Martin, H. (2010). Role of PPAR-gamma in inflammation. Prospects for therapeutic intervention by food components. Mutation Research/Fundamental and Molecular Mechanisms of Mutagenesis, 690(1-2), 57-63.]

Although the bioavailability of these phytochemicals needs to be addressed, the approach demonstrates that many common phytochemicals have the potential to influence PPARγ activity, due to the promiscuous nature of the binding site. Zingerone, the active ingredient in ginger, which is structurally similar to curcumin, is also anti-inflammatory through its ability to increase PPARγ expression.

In some embodiments, the inflammation -targeting nutraceutical is ginsenoside.

Ginsenoside Re ameliorates inflammation by inhibiting the binding of lipopolysaccharide to TLR4 on macrophages [see Lee, In-Ah, et al. “Ginsenoside Re ameliorates inflammation by inhibiting the binding of lipopolysaccharide to TLR4 on macrophages.” Journal of agricultural and food chemistry 60.38 (2012): 9595-9602.] Ginsenoside Rb1 and its metabolite compound K inhibit IRAK-1 activation—The key step of inflammation [see Joh, Eun-Ha, et al. “Ginsenoside Rb1 and its metabolite compound K inhibit IRAK-1 activation—the key step of inflammation.” Biochemical pharmacology 82.3 (2011): 278-286.]

In the preliminary study, ginsenoside Rb1, a main constituent of the root of Panax ginseng (family Araliaceae), and its metabolite compound K inhibited a key factor of inflammation, nuclear transcription factor κB (NF-κB) activation, in lipopolysaccharide (LPS)-stimulated murine peritoneal macrophages. Ginsenoside Rb1 and compound K also significantly inhibited the activation of interleukin-1 receptor-associated kinase-1 (IRAK-1), IKK-β, NF-κB, and MAP kinases (ERK, JNK, and p-38).

In some embodiments, the inflammation -targeting nutraceutical is c-phycocyanin Biliprotein from Spirulina platensis

C-phycocyanin is a selective inhibitor of cyclooxygenase-2 (COX-2) with a very low IC50 COX-2/IC50 COX-1 ratio (0.04). The extent of inhibition depends on the period of preincubation of phycocyanin with COX-2, but without any effect on the period of preincubation with COX-1.[see Reddy, C. Madhava, et al. “Selective inhibition of cyclooxygenase-2 by C-phycocyanin, a biliprotein from Spirulina platensis.” Biochemical and biophysical research communications 277.3 (2000): 599-603.]

In some embodiments, the targeting nutraceutical is a viral -inflammation targeting nutraceutical . In some embodiments, the viral -inflammation targeting nutraceuticals are fucoidans.

Antiviral activity is ensured by the binding of fucoidans to the CD4 glycoprotein on T lymphocytes, an essential immunoglobulin in the infection process of host cells by the viruses [see Chollet, Lucas, et al. “Fucoidans in nanomedicine.” Marine drugs 14.8 (2016): 145.]

The mode of action of the galactofucan was shown to be the inhibition of viral binding and entry into the host cell Furthermore, algal fucoidans have been found to suppress IgE and Th2 cytokine production as shown in recent studies.

Therapeutic activity: In some embodiments, the complex comprises at least one Active Pharmaceutically Ingredient (API). An API is a compound that has a pharmacological (or therapeutic) effect. In some embodiments, the API is a synthetic compound, a natural product, a semi-natural product or a neurochemical. In some embodiments, the API is an active nutraceutical. In some embodiments, at least one API is bound/attached to the outer surface/shell of the NP, contained with the interior volume of the nanoparticle or bound within the NP (within the lipid layer of the NP). In some embodiments, the complex comprises a linker that binds to the ligand and to the API.

The term “active nutraceutical ingradient (ANI)” refers to a compound that is derived or originated from a natural source and has been clinically shown to be beneficial in the prevention and/or treatment of a disease, disorder or condition of a subject suffering therefrom (see also definition in Chao et al. (2012) Nutrition Reviews, 70(7), 373-386). The ANI can be targeted to various systems and disorders, such as the central or peripheral nervous system (such as in Parkinson's disease, Alzheimer disease or multiple sclerosis), neo-vascularization or neoplasmic malignant growth (solid cancer tumors), endocrine system (such as diabetics) or the gastrointestinal system (such as gastric ulcers or IBD).

In some embodiments, the active nutraceutical is a neuro-active nutraceutical:

In some embodiments, at least one neuro-active nutraceutical is selected from polyphenols, including simple polyphenols and phenolic acid derivatives, such as Gallic acid, Curcumin, Ferulic acid, Rosmarinic acid, and Verbascoside. The nutraceutical can be selected from complex polyphenols including Flavonoids (such as Catechin, Epicatechin, EGCG, Kaempferol, Morin, Myricetin or Quercetin), Flavones (such as Apigenin, Scutellarein and Baicalein), Isoflavone (such as Genistein), Anthocyanidins (such as Delphinidine and Proanthocyanidins), Tannins (such as Tannic Acid), Stilbenes (such as Piceatannol, Piceid, Resveratrol, Viniferin), omega fatty acids (such as cicosapentaenoic (EPA) and docosahexaenoic (DHA)) and Lignans (such as Nordihydroguaiaretic acid (NDGA)). The (neuro)-active nutraceutical can also be sugar alcohol like mannitol, sorbitol, and any combinations thereof.

In some embodiments, the neuro-active nutraceutical has anti-oxidative properties and neurprotective properties.

In some embodiments, the active nutraceutical is an onco-active nutraceutical:

In some embodiments, the onco-active nutraceutical can control the growth of cancer cells. In some embodiments, the onco-active nutraceutical is selected from a group consisting of curcumin, oleic acid. In some embodiments the active nutraceutical is a spice extract, selected from ajoene, allicin, allyl isothiocyanate, bergapten, β-carotene, α-sitosterol, canthaxanthin, capsaicin, carnosol, cineole, cinnamaldehyde, citral, crocin, curcumin, diallyl disulfide, diallyl sulfide, diallyl trisulfide, [6]-gingerol, kaempferol, limonene, lutein, myristicin, [6]-paradol, rosmarinic acid, S-allyl cysteine, S-allyl mercapto cysteine, sesamin, sesamolin, shogaol, sulforaphane, terpinene-4-ol, turmerone, and ursolic acid.

In some embodiments, the onco-active nutraceutical is a dye such as an alkannin (or an analog or enantiomer of alkanes). In some embodiments, the onco-active nutraceutical is a plant hormone, such as Jasmonic acid (JA) (shown to induce death in lymphoblastic leukemia cells) or a derivative of, such as Methyl jasmonate (shown to inhibit proliferation in of cancer cell lines).

In some embodiment, the active nutraceutical regulates the BcL-2 protein.

In some embodiments, the targeting nutraceutical is an endo-active nutraceutical:

In some embodiments, the endo-active nutraceutical can regulate the endocrinological system is a polyphenol selected from a group consisting of stilbenes or stilbenoids (such as Piceatannol and Resveratrol). In some embodiments, the active nutraceutical is hormone (such as Insulin).

In some embodiments, the active nutraceutical is a gastro-active nutraceutical, active in the gastrointestinal tract, such as the stomach or intestine.

In some embodiments, the gastro-active nutraceuticals are fat-soluble vitamins (such as A, D, E, and K). In some embodiments, the gastro-active nutraceuticals are lipids, such as polyunsaturated fatty acids (PUFA). The PUFA can be n-6 PUFA arachidonic acid (AA), which is the precursor of inflammatory eicosanoids like prostaglandin E(2) and leukotriene B(4), and the n-3 PUFAs eicosapentaenoic acid (EPA) and docosahexaenoic acid (DHA), which are abundant in fish oils. Lipids and fat-soluble vitamins can be dissolved in the lipid (bi) layer of the NP. In some embodiments, the NP can comprise a combination of Vitamins and PUFAs.

In some embodiments, the gastro-active nutraceutical is a polyphenol such as is Stevia, a food additive extracted from the leaves of Stevia rebaudiana. Its bioactive elements and/or metabolites include steviol, steviol glycosides, mainly rebaudioside A and stevioside. In some embodiments, the polyphenol is a cyclic or linear (curcuminoid) diarylheptanoids, such as curcumin. In some embodiments, the polyphenol is a green tea flavonoid, such as epigallocatechin-3-gallate (EGCG).

Unless otherwise stated, with reference to numerical quantities, the term “about” refers to a tolerance of +25% of the stated nominal value. Unless otherwise stated, all numerical ranges are inclusive of the stated limits of the range.

Reference is now made to a composition for delivering an active pharmaceutical ingredient (API) to a targeted tissue or a targeted cell 22 of a mammalian body, comprising a complex comprising of: at least one nanoparticle 10; at least one API; the API is contained by the nanoparticle 10; at least one nutraceutical ligand 13, the nutraceutical ligand 13 is configured to specifically bind to the targeted tissue or the targeted cell 22; characterized be the API being activated at the targeted tissue or the targeted cell 22 by the mediation of the nutraceutical ligand 13.

Further reference is made to the above-mentioned composition as defined above, further characterized by the composition further comprising a linker 17 that binds the at least one nutraceutical ligand 13 to the at least one nanoparticle 10.

Further reference is made to the above-mentioned composition as defined in any of the above, further characterized by the nutraceutical being a targeting nutraceutical.

Further reference is made to the above-mentioned composition as defined in any of the above, further characterized by the nanoparticle 10 being selected from the group consisting of a liposome, a micelle, a solid-lipid nanoparticle, a cyclodextrin nanoparticle, a dendrimer, a polymeric nanoparticle, a micro/nano-emulsion, and any combinations thereof.

Further reference is made to the above-mentioned composition as defined in any of the above, further characterized by the micelle being a mono-layer or a bi-layer 11 construction.

Further reference is made to the above-mentioned composition as defined in any of the above, further characterized by the nanoparticle 10 being in the range of 10-250 nm.

Further reference is made to the above-mentioned composition as defined in any of the above, further characterized by the API being bound to the nanoparticle 10 via a chemical or a physical bond.

Further reference is made to the above-mentioned composition as defined in any of the above, further characterized by the API 14 being contained within the mono-layer or bi-layer 11, API 15 being contained between layers of the bi-layer 11, or API 16 being bound to an external layer of the mono-layer or bi-layer 11.

Further reference is made to the above-mentioned composition as defined in any of the above, further characterized by the chemical or physical bond being selected from the group consisting of a covalent bond, a hydrogen bond, a wan der Waals bond, a hydrophobic bond, an electrostatic bond, a London bond, a ionic bond, a salt bridge, adsorbtion, and any combination thereof.

Further reference is made to the above-mentioned composition as defined in any of the above, further characterized by the composition being adapted to release the API at the targeted tissue or the targeted cell 22.

Further reference is made to the above-mentioned composition as defined in any of the above, further characterized by the API being selected from a group consisting of a nutraceutical, a small molecule, a food supplement, a protein, a hormone, a peptide, and a fatty acid.

Further reference is made to the above-mentioned composition as defined in any of the above, further characterized by the nutraceutical being an active nutraceutical ingadient (ANI).

Further reference is made to the above-mentioned composition as defined in any of the above, further characterized by the ANI being selected from a group consisting of a neuro-active nutraceutical, an onco-active nutraceutical, an endo-active nutraceutical, a gastro-active nutraceutical, and any combination thereof.

Further reference is made to the above-mentioned composition as defined in any of the above, further characterized by the ANI being curcumin.

Further reference is made to the above-mentioned composition as defined in any of the above, further characterized by the API being generally recognized as safe (GRAS).

Further reference is made to the above-mentioned composition as defined in any of the above, further characterized by the nutraceutical ligand 13 being selected from a group consisting of a neuro-targeting compound, a tumor-targeting compound, a gastric targeting compound, an endo (pancreatic) -targeting compound, a cardiovascular targeting compound, a kidney targeting compound, an inflammation targeting compound, and any combination thereof.

Further reference is made to the above-mentioned composition as defined in any of the above, further characterized by the neuro-targeting compound being selected from a group consisting of caffeine, theobromine (xantheose), paraxanthine, theophylline, 1,3,7-trimethyluric acid, guarana (Paullinia cupana, HBK), phenethylamine (PEA, chocolate), tryptamine (Acacia), and any combinations thereof.

Further reference is made to the above-mentioned composition as defined in any of the above, further characterized by the neuro-targeting compound being selected from a group consisting of baicalein, wogonin, oroxylin A, and any combination thereof.

Further reference is made to the above-mentioned composition as defined in any of the above, further characterized by the nutraceutical ligand 13 being generally recognized as safe (GRAS).

Further reference is made to the above-mentioned composition as defined in any of the above, further characterized by the composition being configured to achieve at least one of the following: a higher concentration of the API at said target tissue; a lower concentration of the API in non-targeted tissue; a higher activity of the API at said target tissue; an extended activity of the API at the target tissue; an increased delivery of said API to a target cell 22.

Further reference is made to the above-mentioned composition as defined in any of the above, the delivery mechanism of the API further being characterized as an active or a passive uptake mechanism.

Further reference is made to the above-mentioned composition as defined in any of the above, further characterized by the uptake mechanism of the API being defined as the uptake of the at least one nanoparticle 10 containing the at least one API, or of the at least one API released from the at least one nanoparticle 10.

Further reference is made to the above-mentioned composition as defined in any of the above, further characterized by the uptake mechanism of the at least one nanoparticle 10 containing the at least one API being endocytosis.

Further reference is made to the above-mentioned composition as defined in any of the above, further characterized by the mode of the endocytosis being selected from the group consisting of receptor-mediated endocytosis, clathrin-mediated endocytosis, non-mediated endocytosis, caveolae, pinocytosis and phagocytosis.

Further reference is made to the above-mentioned composition as defined in any of the above, the active uptake mechanism further characterized as being facilitated by one of the group consisting of transporters, pumps and channels.

Further reference is made to the above-mentioned composition as defined in any of the above, further characterized by the passive uptake mechanism being selected from the group consisting of diffusion, facilitated diffusion and filtration.

Further reference is made to the above-mentioned composition as defined in any of the above, further characterized by the uptake being selected from the group consisting of secondary active transport, coupled transport or co-transport.

### EXAMPLES

### Example 1: Preparation of the Nanoparticle Complex

Liposomes containing targeting nutraceutical covering a core containing an active nutraceutical are manufactured by formulating a self-assembling amphiphilic biocompatible lipid, such as glyceryl monooleate (GMO), loaded with an active nutraceutical and “decorated” with targeting nutraceutical. The liposome can be produced by sonication, extrusion, reverse-phase evaporation, and solvent injection. Production may further include separation of the produced liposomes by size.

Cellular Uptake Study [as described in Ismail et al., 2013. International Journal of Nanomedicine, 8, 393-406]. Cellular uptake of the drug-loaded NPs is essential for attaining ample drug level to elicit a substantial therapeutic response.

ADME (Targeting: Absorption, Distribution and accumulation studies- NPs (liposomes) loaded with a targeted nutraceutical, are marked with a fluorescence dye, or radioactive (3H, 14C) liposomes. The loaded liposomes are injected into the mice (Intravenously, via the gastrointestinal cavity, subcutaneously) and scanned to identify and measure the accumulation of the fluorescence dye in target tissues.

### Example 2: Neurological function

Cell Viability Study are conducted to assess the effect of decorated liposomes on rotenone-induced toxicity, via a cell viability study by 3-(4,5-dimethylthiazol-2-yl)-2,5- diphenyltetrazolium bromide (MTT) assay was performed in PC12 cells. The cytotoxicity study with rotenone should reveal a dose dependency with 2 μg/mL of rotenone causing approximately 50% cell death. Further, to test the protective effect of decorated liposomes on rotenone-induced toxicity, we will expose the PC12 cells to rotenone (2 μg/mL) along with various concentrations of caffeine/baicalein/ wogonin/ oroxylin A mannitol, or both, native or in liposomal nanoformulations.

As depicted in FIG. 3, caffein-decorated SNLs containing curcumin were shown not to be toxic. In Vivo Studies In vivo studies are performed on male albino rats of Wistar strain weighing 260±20 g. Before starting the experiments, animals are allowed to acclimatize for 1 week and had access to rat chow and water ad libitum throughout the study. The experiments are performed in accordance with ethical procedures and policies approved by the Animal Care and Use Committee of the University or private institution faculty, following the 18th World Medical Association General Assembly, Helsinki, June 1964, and updated by the 59th World Medical Association General Assembly, Seoul, October 2008.

Rats are assigned into two groups, each containing twelve animals. Group I rats are administered ultrapure water orally and served as normal control. In group II, rats receive oral decorated (50 mg/kg/day). The rats are tracked and evaluated for 3 months.

Behavioral Experiments [as described at Ismail et al. 2013. International Journal of Nanomedicine, 8 393-406]

Morris water maze—After 3 months, spatial memory is be measured by the Morris water-maze (MWM) test. The water maze consisted of a circular water tank (160 cm in diameter and 35 cm in height), which is divided by four fixed points on its perimeter to four quadrants. It contains an escape platform of 10 cm in diameter of the same color as the rest of the basin (to eliminate any false-positive results due to vision), placed in a constant quadrant of the basin throughout the trials and kept 1.5 cm below the water surface. Rats are placed at a start point in the middle of the rim of a quadrant not containing the escape area with their face to the wall. Animals have four trials per day separated by 10 minutes for 5 successive days, during which the times required to find the hidden platform which are be averaged.

Additional studies include, lipid-based liposomes, restoration of functional deficits in a Rotenone-induced mouse model of PD, measurement of intracellular oxidative stress, induction of Autophagy-Lysosome functions (The autophagy-lysosome pathway (ALP) is a vital mechanism for the removal of abnormal and aggregated proteins, and ample of evidence suggests an impairment in this pathway, which thereby aggravates disease progression), modulation of apoptosis, plasma bioavailability and brain bio-distribution of mannitol loaded and caffeine decorated liposomes, restoration of functional deficits in a Rotenone-induced mouse model of PD among other possible research experiments.

Preliminary results: As depicted in FIGS. 4a-d, after 1 h (FIGS. 4b) and 1.5 h (FIG. 4c-d) of treatment of Human Astrocytes (U-87 MG cell line), a significant increase in the curcumin fluorescence was observed following SLN+Caffeine sample, as compared to the ligand-less SLN control sample and to SLN-less caffeine sample. This indicates that the coating of the sample with caffeine 13 augments the intracellular content of curcumin 14 at 1.5 hours. In addition, as shown in FIG. 4e, blocking of A2R receptors with an antagonist to caffeine, reduces the curcumin content to the same levels as the SLN control (in the concentration of 20 mg/ml). In fluorescent imaging of said cell line, significantly higher curcumin 32 staining can be observed in the SLN 10+Caffeine 13 treatment at 1.5 hours (FIG. 6a), compared to SLN 10 control (FIG. 6b) and untreated cells 30 (FIG. 6c). In untreated cells 30, green color is not detectable. In SLN 10 control cells 30, certain amount of green color is detectable around the nuclei 31 of the cells 30. In caffeine-decorated cells 30, the green color is so strong it even interferes with the blue color of the nucleus 31. Numerical analysis of the fluorescence images will be provided later.

In another cell line comprising dopamine receptors, Human Neuroblastoma cells (SH Sy5Y), as depicted in FIG. 5a, after 1 hour, a significant increase in the curcumin 14 fluorescence was observed following SLN 10+Caffeine 13 sample, as compared to the SLN 10 control sample and to curcumin 14 sample. However, as depicted in FIG. 5b, after 3.5 hours of treatment, the fluorescence of the SLN 10 control sample and the curcumin 14 sample remained at the same levels, as opposed to the SLN 10+caffeine 13 sample, which diminished considerably. This effect will need explanation. However, the above-mentioned results still showcase the influence which ligands 13 have on the uptake of the API 14.

Parkinson's Disease: Evaluation of PD treatments is be evaluated in both 6-OHDA and MPTP Parkinson's disease models, administering the NP to mice (n=8). The mice receive four intraperitoneal (i.p.) injections of MPTP-HCl (18 mg/kg of free base) in saline at 2-h intervals. The intoxicated mice are treated daily with 100 μL NP-based composition comprising a neuro-targeting nutraceutical and a neuro-active nutraceutical from 3 h after the last injection of MPTP via gavage using a gavage needle.

Two types of behavioral experiments are conducted: open field experiment for locomotor activity and a rotarod experiment for feet movement. Locomotor activity is measured 7 days after the last dose of MPTP injection in a Digiscan Monitor (Omnitech Electronics, Inc., Columbus, OH). This Digiscan Monitor records stereotypy and rearing, behaviors that are directly controlled by the striatum, as well as other basic locomotion parameters, such as horizontal activity, total distance traveled, number of movements, movement time, rest time, mean distance, mean time, and center time. Before any insult or treatment, mice are placed inside the Digiscan Infra-red Activity Monitor for 10 min daily and on arotarod for 10 min daily for 3 consecutive days to train them and record their baseline values. Briefly, animals are removed directly from their cages and gently placed nose-first into a specified corner of the open-field apparatus and after release, data acquisition began at every 5 min interval. DIGISCAN software is used to analyze and store horizontal and vertical activity data, which are monitored automatically by infrared beams. In rotarod, the feet movement of the mice is observed at different speeds. To eliminate stress and fatigues, mice are given a 5-min rest interval. Then 7 d after the last dose of MPTP, open field assays and arotarod tests are carried out twice at 6 h intervals on each mouse separately. Locomotor activity measures are assessed after baseline value comparison.

Multiple sclerosis: 6-week old male DA rats (n=8, weight=200 gr±20 gr) are kept at 23±2° C. with a relative humidity of 50% and lights on at 6 a.m. on and off at 6 p.m. Animals are fed ad libitum with a commercial diet with free access to water. EAE disease is induced by immunization of rats with the encephalitogenic emulsion prepared by mixing equal amounts of freshly extracted DA rat spinal cord homogenate (50% w/v in saline) and complete Freund's adjuvant (CFA) containing 7 mg/ ml Mycobacterium tuberculosis. Immunized rats are monitored and neurological scores assessed in an unblinded fashion daily beginning on day 5 post disease induction for clinical symptoms of EAE. Clinical disease is scored for typical signs according to the following scale: 0, no clinical signs; 1, flaccid tail; 2, hind limb weakness; 3, hind limb paresis; 4, complete bilateral hind limb paralysis; 5, death due to EAE]. The NP-based composition comprising a neuro-targeting nutraceutical and a neuro-active nutraceutical is suspended in carboxymethylcellulose made up to 0.5% in water to which Tween 80 is added to reach a final concentration of 0.6% and is administered orally daily for 34 days. For therapeutic treatment, monitoring animals for signs of clinical disease starts five days following disease induction.

Alzheimer's diseases: Experiments are carried out in a double transgenic animal model of AD ( overexpressing human APP with the London mutation (V717I) and human PSI bearing the A246E mutation). 6-month old mice (n=8, weight=animals are treated by intra-cerebroventricular (i.c.v.) infusion and intraperitoneal (i.p.) injection of the NP-based composition comprising a neuro-targeting nutraceutical and a neuro-active nutraceutical for 8 wk. The immunological reaction to the treatment is studied in the i.p. treated animals. Histological analysis is conducted for amyloid plaques, amyloid load in the cerebral cortex and hippocampus, respectively.

### Example 3: Oncological Function

Cancer: In vitro screening of various cancer types, and then test the decorated liposomes in IN VIVO mouse models—xenograft mouse mode for brain glioma, colon cancer, breast cancer, tumor cells (colon cancer) are grown subcutaneously in immune-deficient hosts (severe combined immune deficient (SCID) mice) for 14 days. The tumor is measured and the NP-based composition comprising an onco-target-nutraceutical and an onco-active nutraceutical is injected. The tumor is evaluated for 14 days following treatment.

Glycolysis blocker: to test the effect of the NP on tumor growth in vivo, 6-week-old male SCID mice are injected with B16 melanoma cells (or other cancer cells) subcutaneously (1×10⁶cells per mouse, 6 mice per group). After the xenografts were established, the tumor-bearing mice are administered with an NP-based composition comprising an onco-target-nutraceutical and an onco-active nutraceutical is injected via intraperitoneal injection. The mice are evaluated for 14 days.

### Example 4: Endocrine Function

Diabetes: chemical models have been shown to be effective for the evaluation of glucose-lowering treatments. STZ [2-deoxy-2-(3-(methyl-3-nitrosoureido)-D-glucopyranose] is administered (via i.p. or i.v.) and enters the pancreatic beta-cell through the Glut-2 transporter and causes alkylation of the DNA (Szkudelski, 2001). Subsequent activation of PARP leads to NAD+depletion, a reduction in cellular ATP and subsequent inhibition of insulin production (Sandler and Swenne, 1983). A single dose of STZ (150 mg/kg) is administered to mice (n=8). 24 hours following STZ administration, the NP comprising a pancreatic-targeting nutraceutical and a pancreatic-active nutraceutical is administered via i.p. to half of the mice and the blood-glucose levels of both groups are tracked for 10 days.

Wistar-Bratislava albino male rats (n=8, weight 225 gr±25 gr) are kept in polypropylene cages at constant temperature (24+2° C.), 60±5% humidity, and light-dark regime. The rats receive unrestricted access to food and water. The NP comprising a pancreatic-targeting nutraceutical and a pancreatic-active nutraceutical is administered via i.p. 30 min before STZ administration, according to Porfire et al. After 72 h, fasting plasma glucose is checked, and the animals with plasma glucose values of more than 200 mg/dL are considered diabetic (and included in the study).

Pancreatic lipase activity assay: Commercial pancreatic lipase is prepared according to McDougall et al. Pancreatic lipase is prepared and the purity of the lipase and co-lipase is estimated by SDS-PAGE analysis. Protein concentration is determined by Bradford. Pancreatic lipase substrate (pNPL, 0.08% mass per volume) is dissolved in 5 mM sodium acetate solution (pH=5.0, 1% Triton X-100). An NP-based composition comprising a pancreatic-targeting nutraceutical and a pancreatic-active nutraceutical dissolved at three different concentrations in methanol. An Orlistat solution is prepared at 1.2 mg/mL in methanol. Stability of the compounds in the solvent is tested for the time of assay by measuring their UV-Vis spectra.

### Example 5: Gastrointestinal Function

Inflammatory Bowel Disorder (IBD) (TNBS model): 6-8-year-old Wister rats (240-280g) (n=10) are dosed with 100 mg/kg following a period (24 h) of fasting, to give an average Wallace score of 5. The mice are evaluated 3 and 7 days after dosing. 7 days following TNBS dosing, the mice are treated with an NP-based composition comprising a gastro-targeting nutraceutical and a gastro-active nutraceutical and evaluated for 14 days.

Fatty liver: Non-alcoholic fatty liver disease (NAFLD) is emerging as a common medical problem. Mice are feed a diet that consists of considerable amounts of sucrose (40% of energy), only moderately enriched with fat (10%) and deficient in methionine and choline. Male wild-type (WT) mice C57B1/6 are fed the MCD diet (MP Biomedicals, #960439; n=15 mice) for 8 weeks. The diet started is started at 12 weeks of age, with the mice treated with an NP-based composition comprising a target-nutraceutical and an active nutraceutical after 8 weeks on the diet (20 week age). Inflammation is tracked for 3 weeks.

Gastric ulcers: Balbc mice (n=8) are infected with H. pylori (Sydney strain) with 0.2 cc of inoculum intragastrically using a 24 gauge×1 in. straight stainless steel feeding tube attached to a 1 cc syringe (approximately 2×108 organisms). The mice receive a total of 3 doses, 1 per day and each separated by 1-2 days. 21 days following infection, the mice are treated with an NP-based composition comprising a gastro-targeting nutraceutical and a gastro-active nutraceutical. The mice are evaluated for 15 days.

Obesity: Zucker Diabetic Fatty (ZDF) rats (n=8, average weight 135 gr±5 gr) were maintained under pathogen-free conditions at 23±0.5° C. and 55%+10% relative humidity with a 12 h light-dark cycle and were provided with water and a commercial diet and are evaluated by treating the mice with an NP-based composition comprising a gastro-targeting nutraceutical and a gastro-active nutraceutical for 14 weeks and tracking the weight.

Enzymatic assay of lipase from Human Pancreas: Obesity is a disarray of energy balance and primarily considered as a disorder of lipid metabolism. A growing number of enzymes involved in lipid metabolic pathways are being identified and characterized. They represent a rich pool of potential therapeutic targets for obesity. Inhibition of PL (triacylglycerol acyl hydrolase), the principal lipolytic enzyme, synthesized and secreted by is one of the approaches for the development of newer anti-obesity drugs. Tetrahydrolipstatin (Orlistat), a commercial anti-obesity drug, is a known pancreatic lipase inhibitor. The NP is further characterized by in-vitro lipase activity to access their anti-obesity potential. Measurement of pancreatic lipase activity in-vitro Lipase activity is determined by measuring the rate of release of oleic acid from triolein.

### Example 6: Kidney Function

Kidney functionality: 8-week-old female Wistar rats (n=12, weighing 180 gr±20 gr) are housed in single metabolic cages at constant room temperature (20° C.) and humidity (75%) under a controlled light/dark cycle. The rats are fed a standard chow diet and animals had free access to drinking water. The rats are treated with an NP-based composition comprising a nephrotic-targeting nutraceutical and a nephrotic-active nutraceutical at day 0. 4 rats are sacrificed every 7 days for the morphological studies of the kidneys.

Nephritis: the study is conducted in normotensive female Wistar rats (n=8, weights of 210 gr±20 gr). Immune complex nephritis is induced with the rats receiving an initial subcutaneous injection of 5 mg of incomplete Freund's adjuvant (CFA). At week 3, a second dose is given in incomplete Freund's adjuvant. At week 4, daily intraperitoneal administration of 10 mg ovalbumin is started. Proteinuria should appear, starting at the 9th week. When proteinuria reaches 20 to 50 mg/day, animals are treated orally with NP-based composition comprising a nephrotic-targeting nutraceutical and a nephrotic-active nutraceutical. Three weeks after the onset of proteinuria. animals are sacrificed, blood collected and kidneys removed.

1.-27. (canceled)

28. A composition for delivering an active pharmaceutical ingredient (API) to a targeted tissue or a targeted cell of a mammalian body, comprising a complex comprising of:
a) at least one nanoparticle;
b) at least one active ingredient, said active ingredient is contained by said nanoparticle; and
c) at least one nutraceutical ligand, said nutraceutical ligand is configured to specifically bind to said targeted tissue or said targeted cell;
wherein said active ingredient is activated at said targeted tissue or at said targeted cell by the mediation of said nutraceutical ligand.

29. The composition of claim 28, wherein said composition further comprises a linker that binds said at least one nutraceutical ligand to said at least one nanoparticle.

30. The composition of claim 29, wherein wherein said nutraceutical ligand is a targeting nutraceutical ligand.

31. The composition of claim 28, wherein said nanoparticle is selected from the group consisting of a liposome, a mono-layer micelle, a bi-layer micelle, a solid-lipid nanoparticle, a cyclodextrin nanoparticle, a dendrimer, a polymeric nanoparticle, a micro/nano-emulsion, and any combinations thereof.

32. The composition of claim 28, wherein the diameter of said nanoparticle is in the range of 10-250 nm.

33. The composition of claim 31, wherein said active pharmaceutical ingredient (API) is bound to said nanoparticle via a chemical or a physical bond, and at least one of the following is true:
a) said API is contained within said mono-layer micelle or said bi-layer micelle, contained between layers of said bi-layer micelle, or bound to an external layer of said mono-layer micelle or said bi-layer micelle;
b) said chemical or physical bond is selected from the group consisting of a covalent bond, a hydrogen bond, a wan der Waals bond, a hydrophobic bond, an electrostatic bond, a London bond, a ionic bond, a salt bridge, adsorbtion, and any combination thereof; or
c) said composition is adapted to release said API at said targeted tissue or at said targeted cell.

34. The composition of claim 28, wherein said API is selected from the group consisting of a nutraceutical, a small molecule, a food supplement, a protein, a hormone, a peptide, and a fatty acid.

35. The composition of claim 34, wherein said said nutraceutical is an active nutraceutical ingredient (ANI) selected from the group consisting of a neuro-active nutraceutical, an onco-active nutraceutical, an endo-active nutraceutical, a gastro-active nutraceutical, and any combination thereof.

36. The composition of claim 35, wherein said ANI is curcumin.

37. The composition of claim 28, wherein said API is generally recognized as safe (GRAS).

38. The composition of claim 28, wherein said nutraceutical ligand is selected from the group consisting of a neuro-targeting compound, a tumor-targeting compound, a gastric targeting compound, an endo (pancreatic) -targeting compound, a cardiovascular targeting compound, a kidney targeting compound, an inflammation targeting compound, and any combination thereof.

39. The composition of claim 38, wherein said neuro-targeting compound is selected from the group consisting of caffeine, theobromine (xantheose), paraxanthine, theophylline, 1,3,7-trimethyluric acid, guarana (Paullinia cupana, HBK), phenethylamine (PEA, chocolate), tryptamine (Acacia), and any combinations thereof.

40. The composition of claim 38, wherein said neuro-targeting compound is selected from a group consisting of baicalein, wogonin, oroxylin A, and any combination thereof.

41. The composition of claim 38, wherein said nutraceutical ligand is generally recognized as safe (GRAS).

42. The composition of claim 28, wherein said composition is configured to achieve at least one of the following:
a) a higher concentration of said API at said target tissue;
b) a lower concentration of said API in non-targeted tissue;
c) a higher activity of said API at said target tissue;
d) an extended activity of said API at said target tissue; or
e) an increased delivery of said API to a target cell.

43. The composition of claim 28, wherein the delivery mechanism of said API is characterized as being an active or a passive uptake mechanism.

44. The composition of claim 43, wherein said uptake mechanism of said API is defined as the uptake of said at least one nanoparticle containing said at least one API, or of said at least one API released from said at least one nanoparticle.

45. The composition of claim 43, wherein said uptake mechanism of said at least one nanoparticle containing said at least one API is endocytosis, said endocytosis selected from the group consisting of receptor-mediated endocytosis, clathrin-mediated endocytosis, non-mediated endocytosis, caveolae, pinocytosis and phagocytosis.

46. The composition of claim 43, wherein said active uptake mechanism is characterized as being facilitated by one of the group consisting of transporters, pumps and channels.

47. The composition of claim 43, wherein said uptake mechanism is selected from the group consisting of diffusion, facilitated diffusion, filtration secondary active transport, coupled transport and co-transport.
